(19) United States
(12) Patent Application Publication
(10) Pub. No.: US 2018/0235643 A1
(43) Pub. Date: Aug. 23, 2018

(54) BONE FUSION SYSTEM AND METHOD

(71) Applicant: DSAN Technology, LLC, Boca Raton, FL (US)

(72) Inventors: Robert E. Lins, Boca Raton, FL (US); Dennis Ty, Jupiter, FL (US)

(21) Appl. No.: 15/887,109

(22) Filed: Feb. 2, 2018

Related U.S. Application Data
(63) Continuation-in-part of application No. 14/882,792, filed on Oct. 14, 2015.
(60) Provisional application No. 62/453,658, filed on Feb. 2, 2017.
(60) Provisional application No. 62/464,570, filed on Feb. 28, 2017.
(60) Provisional application No. 62/095,120, filed on Dec. 22, 2014.
(60) Provisional application No. 61/118,759, filed on Dec. 1, 2008.

(51) Int. Cl. A61B 17/17; A61B 17/88; A61F 2/46

(52) U.S. Cl. CPC A61B 17/1775; A61B 17/1764; A61B 17/1782; A61F 2/461; A61B 17/8816; A61F 2/4606; A61F 2/4611; A61B 17/1757

(57) ABSTRACT

Joint fixation systems and methods enabling machining of a plurality of overlapping bores in an articulated skeletal joint with at least one bore on each side of abutting surfaces at the joint. An implant is secured to bones on opposite sides of the joint to immobilize the joint in order to effect fusion of the joint. A jig is used to effect a desired location of the overlapping bores during machining.

### PRIORITY CLAIM

In accordance with 37 C.F.R. 1.76, a claim of priority is included in an Application Data Sheet filed concurrently herewith. Accordingly, the present invention claims the benefit of priority to U.S. Provisional Patent Application No. 62/453,658, entitled “JOINT FUSION SYSTEMS AND METHODS FOR THE FOOT AND ANKLE”, filed Feb. 2, 2017; and to U.S. Provisional Patent Application No. 62/464,570, entitled “ADJACENT FOR INTRA BONE FUSION DEVICE AND IMPLANT”, filed Feb. 28, 2017. This application is also a Continuation-in-Part of Application Ser. No. 14/882,792, entitled “SACROILIAC JOINT FUSION SYSTEMS AND METHODS”, filed Oct. 14, 2015, which claims the benefit of priority to U.S. Provisional Patent Application No. 62/095,120, entitled “SACROILLIAC JOINT FUSION SYSTEMS AND METHODS, filed Dec. 22, 2014; and to U.S. Provisional Patent Application No. 61/118,759, entitled “SACROILIAC JOINT FUSION SYSTEMS AND METHODS”, filed Feb. 20, 2015. The contents of the above referenced applications are incorporated herein by reference.

### FIELD OF THE INVENTION

The present invention relates generally to a joint fusion system and method for use to immobilize an articulating joint, such as a highly articulating ankle joint or a semi mobile joint, such as a cervical spine joint, to allow adjacent bones to fuse together.

### BACKGROUND OF THE INVENTION

The sacroiliac joint is the joint between the sacrum and the ilium of the pelvis. The sacrum and the ilium are joined by ligaments. The sacrum supports the spine and is supported, in turn, by the ilium on each side. The sacroiliac joint is a synovial joint, with articular cartilage and irregular elevations and depressions that produce interlocking of the sacrum and the ilium.

Pain associated with the sacroiliac joint can be caused by traumatic fracture, dislocation of the pelvis, degenerative arthritis, sacroiliitis, a degenerative condition or inflammation of the sacroiliac joint, osteitis condensans ilii, or the like. Sacroiliac joint fusion is often indicated as a surgical treatment for such conditions. Sacroiliac joint fusion can be performed via an anterior approach, a posterior approach, or a lateral approach, and typically involves the placement of a fixation assembly, an implant, and/or one or more screws. Significant problems exist, especially when sacroiliac joint fusion is performed via an open surgical procedure, for example.

Open surgical procedures require general anesthesia and can involve considerable operative time, recovery time, hospitalization, and pain due to significant soft tissue damage. Damage to blood vessels and nerves is also possible. Specifically, the placement of a fixation assembly, an implant, and/or one or more screws can cause damage to the lumbosacral neurovascular elements and/or delayed union of the sacroiliac joint. In a worst case scenario, this can require revision or removal surgery.

Minimally-invasive surgical procedures are technically more difficult and require multiplanar fluoroscopy/radiography of the articular surfaces of the sacroiliac joint, for example. Again, the placement of a fixation assembly, an implant, and/or one or more screws can cause damage to the lumbosacral neurovascular elements and/or delayed union of the sacroiliac joint. Further, sacral anomalies can lead to mal-placement of the implant, leading to damage to the surrounding structures.

In both open and minimally-invasive surgical procedures, insufficient amounts of the articular surfaces and/or the cortical surfaces of the sacroiliac joint may be removed to relieve pain in the sacroiliac joint. Likewise, insufficient amounts of the articular surfaces and/or the cortical surfaces of the sacroiliac joint may be engaged by the fixation assembly, the implant, and/or the one or more screws to ensure adequate stabilization and/or fusion. The failure to adequately stabilize and/or fuse the sacroiliac joint can result in a failure to relieve the condition being treated. Mal-alignment of the sacroiliac joint is a similar problem and can lead to increased pain.

Thus, what are still needed in the art are improved sacroiliac joint fusion systems and methods that provide adequate visualization of and access to the sacroiliac joint, provide very predictable and consistent results easily and efficiently, provide adequate stabilization and/or fusion of the sacroiliac joint, as well as optional distraction and/or translation, if desired, and minimize surgical time, thereby eliminating the problems described above.

In a second embodiment, an articulating joint, such as an ankle joint, a foot bone joint, hand joint, knee joint and joints in the cervical spine, permit relative movement between two adjacent bones, such as in the case on the ankle, the tibia and the talus (also known as an astragalus), and occasionally need to have the adjacent bones fused together to form an immobile joint. The need for such fusion can vary and may be due to joint deterioration or injury. Joints are classed as mobile, an example of which is a highly articulating joint that can pivot 20° or more between the bones on opposite sides of the joint during normal usage, and semi mobile which articulate less, such as cervical spine joints and midfoot joints. During normal articulation, such joints can carry significant loads, presenting special difficulty to effect immobilization while the bones forming the joints are fused together at the joint.

Surgical procedures used to effect fusion can be an open surgical procedure or a minimally invasive surgical procedure. In either type of procedure, it is common to remove material from the joint to allow adjacent bone surfaces to contact one another so that fusion can occur. During the fusion, which can take months to accomplish, the bones forming the joint cannot move relative to one another, lest the fusion process fail or even partially fail.

Joint fusion surgery is an orthopedic operation that is typically used for relieving pain associated with degeneration of the joint due to such maladies as arthritis, deformity, injury, infection and the like and may also be used to relieve pain associated with deterioration of the joint or to take pressure off of a nerve by stabilizing the joint. The surgical procedure generally involves removal of worn-out portions of the joint, after which the bones comprising the joint are immobilized from movement relative to one another using an external device known as a fixator or an intramedullary device. In other case, temporary interoperative external fixators may or may not be utilized depending on the joint. Fixators typically require later surgery to remove them. Current intramedullary devices tend to be complex in structure and difficult to install surgically.

The present invention overcomes the difficulties of the prior art by providing an implant that is contained within the bone structure, and it need not be removed after fusion is accomplished.

### Description of the Prior Art

There are numerous patents relating to both fixator devices and intramedullary devices. U.S. Pat. No. 9,125,695 to Early, et al., is directed to an intramedullary device that is permanently installed and very complex in structure. U.S. Pat. No. 9,161,796 to Chiodo is directed to a fixator that is attached to the side of the tibia and talus, requiring subsequent removal after use. It is not clear from the disclosure how long this device stays on after surgery is complete.

### SUMMARY OF THE INVENTION

The present invention relates to a method and system for holding an articulating joint against pivoting to allow adjacent bones to fuse together after surgery.

Accordingly, it is a primary objective of the present invention to provide a system for machining a series of overlapping bores in bones on both sides of the joint by drilling to receive an implant that will fix the bones forming the joint against relative movement during joint fusion.

It is a further objective of the present invention to install an implant that can pull the bones forming the joint together.

It is yet another objective of the present invention to provide means to align the bones and hold them in a desired orientation relative to one another for machining the overlapping bores that receive the implant.

It is a still further objective of the invention to provide an implant that can be secured to one or more of the joint bones with one or more mechanical fasteners.

Other objects and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

The present invention is illustrated and described herein with reference to the various drawings, in which like reference numbers are used to denote like system components/method steps, as appropriate, and in which:

FIG. 1A is a side view of one exemplary embodiment of the portal tube of the present invention;

FIG. 1B is a top perspective view of the portal tube illustrated in FIG. 1A;

FIG. 1C is a bottom perspective view of the portal tube illustrated in FIG. 1A;

FIG. 2A is a perspective view of one exemplary embodiment of the drill guide tube of the present invention;

FIG. 2B is a bottom perspective view of the drill guide tube illustrated in FIG. 2A;

FIG. 2C is a top perspective view of the drill guide tube illustrated in FIG. 2A;

FIG. 3 is a perspective view of one exemplary embodiment of the portal tube and the drill guide tube of the present invention in an assembled configuration;

FIG. 4A is a side perspective view of one exemplary embodiment of the drill guide of the present invention;

FIG. 4B is a bottom perspective view of the drill guide illustrated in FIG. 4A;

FIG. 4C is a top perspective view of the drill guide illustrated in FIG. 4A;

FIG. 5A is a perspective view of one exemplary embodiment of the portal tube, the drill guide tube, and the drill guide of the present invention in an assembled configuration;

FIG. 5B is a top view of one exemplary embodiment of the portal tube, the drill guide tube, and the drill guide of the present invention in an assembled configuration;

FIG. 6A is a schematic diagram of an exemplary drilling pattern that can be utilized in conjunction with/provided by the present invention;

FIG. 6B is a schematic diagram of an exemplary drilling pattern that can be utilized in conjunction with/provided by the present invention;

FIG. 6C is a schematic diagram of an exemplary drilling pattern that can be utilized in conjunction with/provided by the present invention;

FIG. 6D is a schematic diagram of an exemplary drilling pattern that can be utilized in conjunction with/provided by the present invention;

FIG. 6E is a schematic diagram of an exemplary drilling pattern that can be utilized in conjunction with/provided by the present invention;

FIG. 6F is a schematic diagram of an exemplary drilling pattern that can be utilized in conjunction with/provided by the present invention;

FIG. 6G is a schematic diagram of an exemplary drilling pattern that can be utilized in conjunction with/provided by the present invention;

FIG. 7A is a schematic diagram of another exemplary drilling pattern that can be utilized in conjunction with/provided by the present invention;

FIG. 7B is a schematic diagram of another exemplary drilling pattern that can be utilized in conjunction with/provided by the present invention;

FIG. 8 is a schematic diagram of a further exemplary drilling pattern that can be utilized in conjunction with/provided by the present invention;

FIG. 9A is a perspective view of one exemplary embodiment of the implant guide tube of the present invention;

FIG. 9B is a top perspective view of one exemplary embodiment of the implant guide tube of the present invention;

FIG. 9C is a bottom perspective view of one exemplary embodiment of the implant guide tube of the present invention;

FIG. 10 is a perspective view of one exemplary embodiment of the portal tube and the implant guide tube of the present invention in an assembled configuration;

FIG. 11A is a side view of one exemplary embodiment of the implant of the present invention;

FIG. 11B is a side view of one exemplary embodiment of the implant of the present invention;

FIG. 12A is a perspective view of one exemplary embodiment of the implant insertion tool of the present invention;

FIG. 12B is a perspective view of one exemplary embodiment of the implant insertion tool of the present invention;

FIG. 13 is a side view of an ankle joint in a foot with a pair of implants installed, illustrating a second embodiment of the present invention;

FIG. 14 is a plan view of a drilling jig for use with the second embodiment;

FIG. 15 is a plan view of a modified form of drilling jig for use with the second embodiment;

FIG. 16 is a perspective view of an implant for use with the invention's second embodiment;

FIG. 17 is a perspective view of a modified form of implant adapted for additional mechanical securement to the joint bones;

FIG. 18A is a perspective view of a device usable to help align the joint bones for bore machining;

FIG. 18B is a perspective view of the device illustrated in FIG. 18A showing the bones aligned;

FIG. 19 illustrates another form of drill guide in top plan view.

FIG. 20 is a side view of a joint where a gap between adjacent bones on opposite sides of the joint is wedge shaped;

FIG. 21 is a side view of the joint of FIG. 20, showing an implant in position in the joint; and

FIG. 22 is an enlarged fragmentary side view of the joint of FIG. 20, showing the implant held in place mechanically.

### DETAILED DESCRIPTION OF THE INVENTION

In various exemplary embodiments of the first embodiment of the invention, the present invention provides a portal tube, a drill guide tube, a drill guide, an implant guide tube, an implant, and related instrumentation for fusing or otherwise securing a sacroiliac or other joint via a minimally-invasive or open surgical procedure. These sacroiliac joint fusion systems and methods provide superior visualization of and access to the sacroiliac joint, provide very predictable and consistent results easily and efficiently, provide superior stabilization and/or fusion of the sacroiliac joint, as well as distraction and/or translation, if desired, and minimize surgical time, thereby eliminating the problems described above.

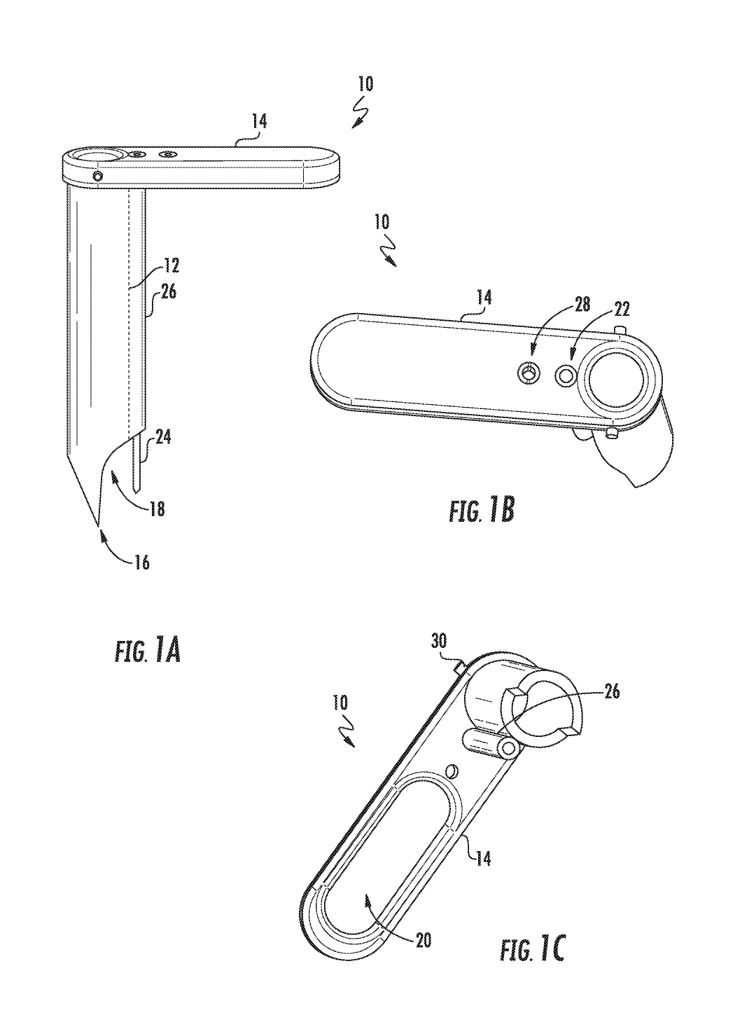

Referring now specifically to FIGS. 1A-1C, in one exemplary embodiment, the portal tube 10 includes a cannulated access tube 12 and a handle 14 coupled to the proximal end of the access tube 12. The handle 14 is used to manipulate the access tube 12, as well as to secure other components inside the access tube 12. All components can be keyed accordingly. The distal end of the access tube 12 includes an angled end 16, optionally including a cut away 18 that is configured and shaped to engage the sacroiliac joint, or another joint, such that the access tube 12 is held in proper alignment, without penetrating too deeply. Preferably, the access tube 12 is made of a surgically compatible metal or plastic, and has a length of between about 120 and about 150 mm, an external diameter of between about 20 and about 25 mm, and an internal diameter of between about 15 and about 20 mm. The handle 14 is a generally paddle shaped component and may include a recess 20, finger contours, etc. The handle 14 includes a first port 22 through which a guide pin 24 is inserted. The access tube 12 includes a corresponding cannulated channel 26 through which the guide pin 24 passes. Preferably, the guide pin 24 includes a shoulder stop (not illustrated) at its proximal end, such that penetration of the guide pin 24 through the channel 26 is limited, and protrudes a predetermined distance beyond the distal end of the channel 26. This protruding portion of the guide pin 24 includes a drill bit (not illustrated), or is threaded such that it can penetrate into the bony structure on one side of the joint, thereby securing the access tube 12 to the joint in the proper alignment. The handle 14 also includes a second port 28 that is configured to receive a corresponding pin associated with other components secured inside the access tube 12, thereby preventing the relative rotation of these components with respect to the access tube 12. Finally, the access tube 12 or handle 14 includes one or more guide pins 30 that are operable for visualizing the alignment of the access tube 12 with respect to the joint in an open or minimally-invasive surgical procedure, optionally under fluoroscopy/radiography. Specifically, these guide pins 30 can be aligned with the joint between the sacrum and the ilium, for example. In general, the access tube 12 acts as a guide relative to the joint in an open surgical procedure or a portal to the joint in a minimally-invasive surgical procedure.

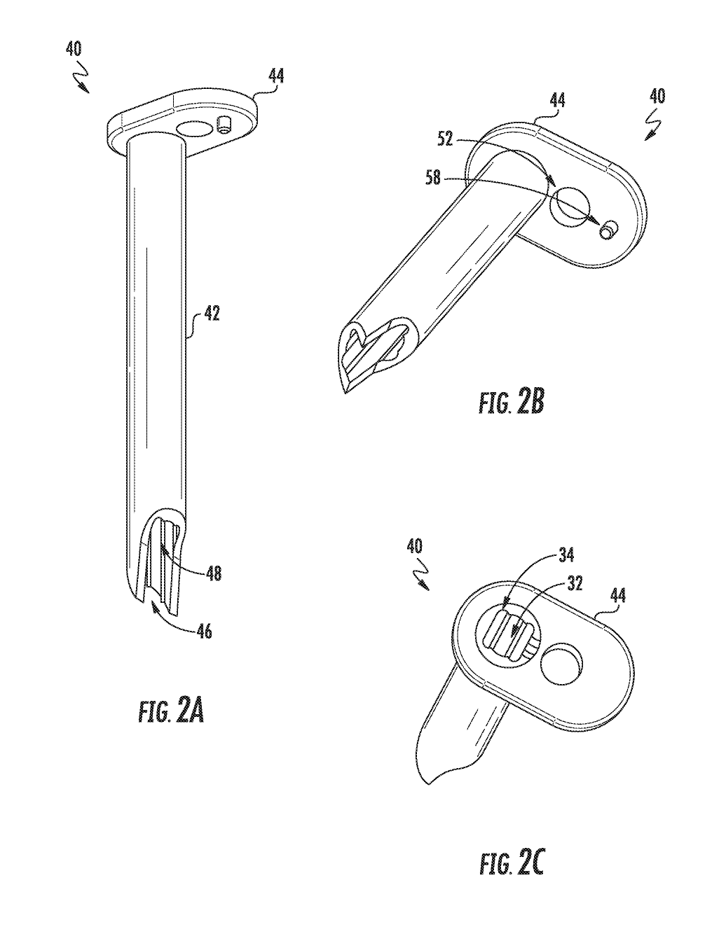

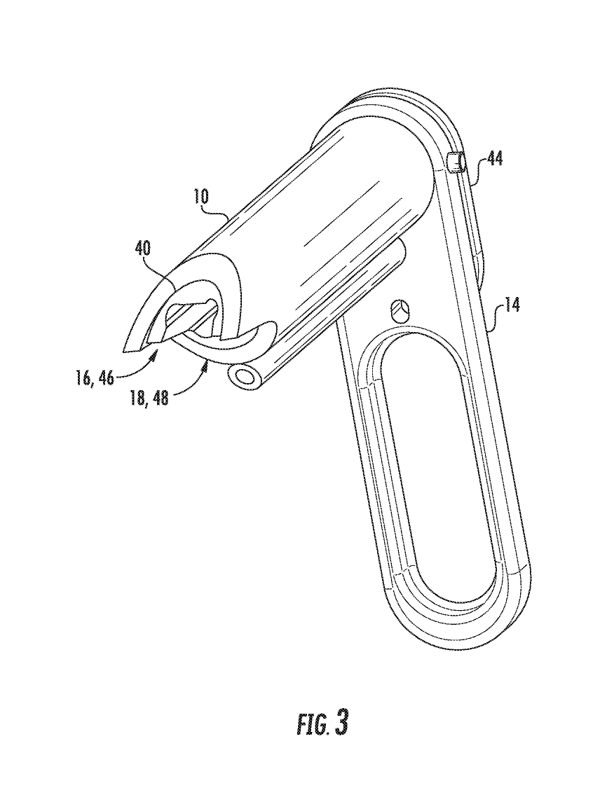

Referring now specifically to FIGS. 2A-2C, in one exemplary embodiment, the drill guide tube 40 includes a cannulated drill tube 42 and a handle 44 coupled to the proximal end of the drill tube 42. In this exemplary embodiment, the drill tube 42 defines a central drill aperture 32 with a plurality of smaller corner drill apertures 34 disposed about the periphery of the central drill aperture 32 in a generally square configuration. This and other exemplary configurations are described in greater detail below. However, all of the drill apertures are configured to receive elongate drill bits (not illustrated) through the drill tube 42, such that a predetermined bore pattern can be drilled into and across the bony structures of the joint, providing a tailored void for receiving an implant, also described in greater detail below. The handle 44 is used to manipulate the drill tube 42, as well as to secure the drill tube 42 inside the access tube 12 (FIG. 1A). The distal end of the drill tube 42 includes an angled end 46 defining one or more points, optionally including a cut away 48, that is configured and shaped to engage the sacroiliac joint, or another joint, such that the drill tube 42 is held in proper alignment, without penetrating too deeply. The angled end 46/cut away 48 of the drill tube 42 is preferably conformal with the angled end 16/cut away 18 (FIG. 1A) of the access tube 12 when the drill tube 42 is inserted into the access tube 12. Preferably, the drill tube 42 is made of a surgically compatible metal or plastic, and has a length of between about 120 and about 150 mm, an external diameter of between about 15 and about 20 mm, and an internal diameter of between about 3 and about 10 mm. The handle 44 is a generally paddle shaped component and may include a recess, finger contours, etc. The handle 44 includes a first port 52 that is configured to receive the shoulder stop of the guide pin 24 (FIG. 1A). The handle 44 also includes a pin 58 that is configured to engage the corresponding second port 28 (FIG. 1B) of the handle 14 (FIG. 1A) of the access tube 12 when the drill tube 42 is inserted into the access tube 12, thereby preventing the relative rotation of the drill tube 42 with respect to the access tube 12. In general, the drill tube 42 acts as a drill guide relative to the joint in an open surgical procedure or a minimally-invasive surgical procedure.

FIG. 3 illustrates the portal tube 10 and the drill guide tube 40 in an assembled configuration, highlighting that the angled end 46/cut away 48 of the drill guide tube 40 is conformal with the angled end 16/cut away 18 of the portal tube 10 when the drill guide tube 40 is inserted into the portal tube 10. The orientation of the handles 14 and 44 is also coincident.

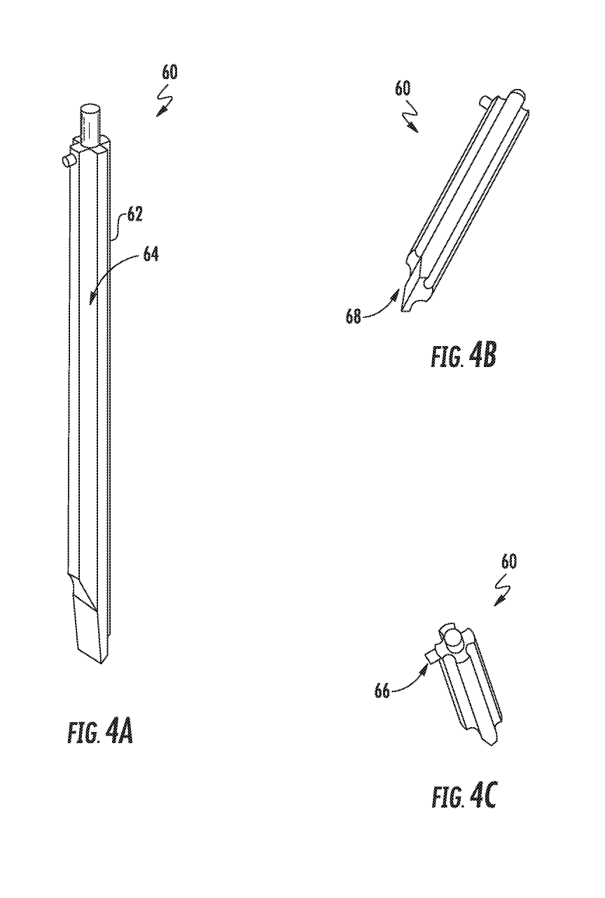

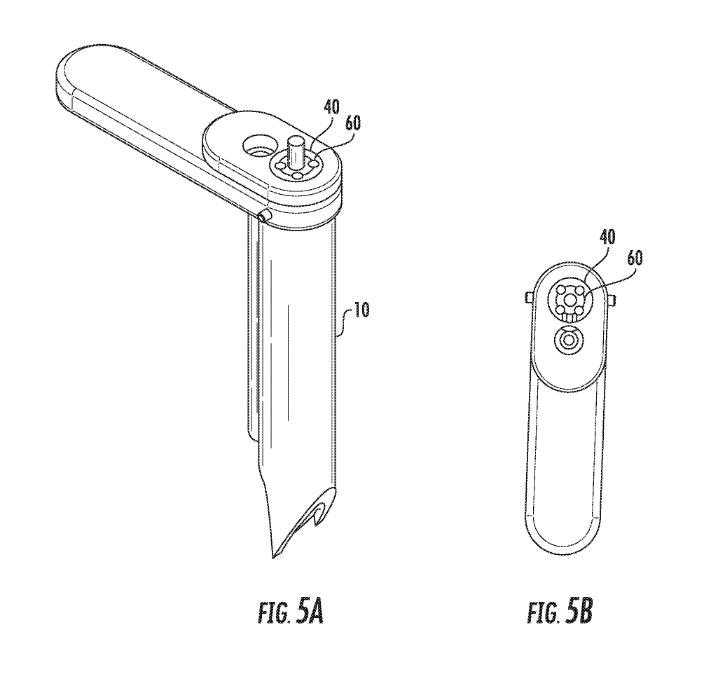

Referring now specifically to FIGS. 4A-4C, in one exemplary embodiment, the drill guide 60 includes an elongate shaft 62 defining a plurality of drill bit receiving channels 64 disposed around the periphery and along the major axis thereof. The elongate shaft 62 is disposed within the drill guide tube 40 (FIGS. 2A-2C and 3), which is disposed within the portal tube 10 (FIGS. 1A-1C and 3). A key 66 protrudes from the proximal end of the elongate shaft 62 and is configured to engage a corresponding recess (not illustrated) manufactured into the handle 14 or 44 of either the portal tube 10 or the drill guide tube 40 or the proximal end of the portal tube 10 or the drill guide tube 40, thereby preventing rotation of the elongate shaft 62 within the drill guide tube 40. In the exemplary embodiment illustrated, the elongate shaft 62 defines four drill bit receiving channels 64 disposed evenly around the periphery and along the major axis of the elongate shaft. Preferably, the drill guide 60 is made of a surgically compatible metal or plastic, and has a length of between about 120 and about 150 mm and an external diameter of between about 15 and about 20 mm. Each of the drill bit receiving channels 64 has a diameter of between about 2.5 and about 5 mm. Optionally, the distal end of the drill guide 60 includes an angled end 68 that is conformal with the angled end 16/cut away 18 (FIGS. 1A, 1C and 3) of the access tube 12 (FIG. 1) and the angled end 46/cut away 48 (FIGS. 2A, 2B and 3) of the drill tube 42 when the drill tube 42 is inserted into the access tube 12.

FIGS. 5A-5B illustrates the portal tube 10, the drill guide tube 40, and the drill guide 60 in an assembled configuration.

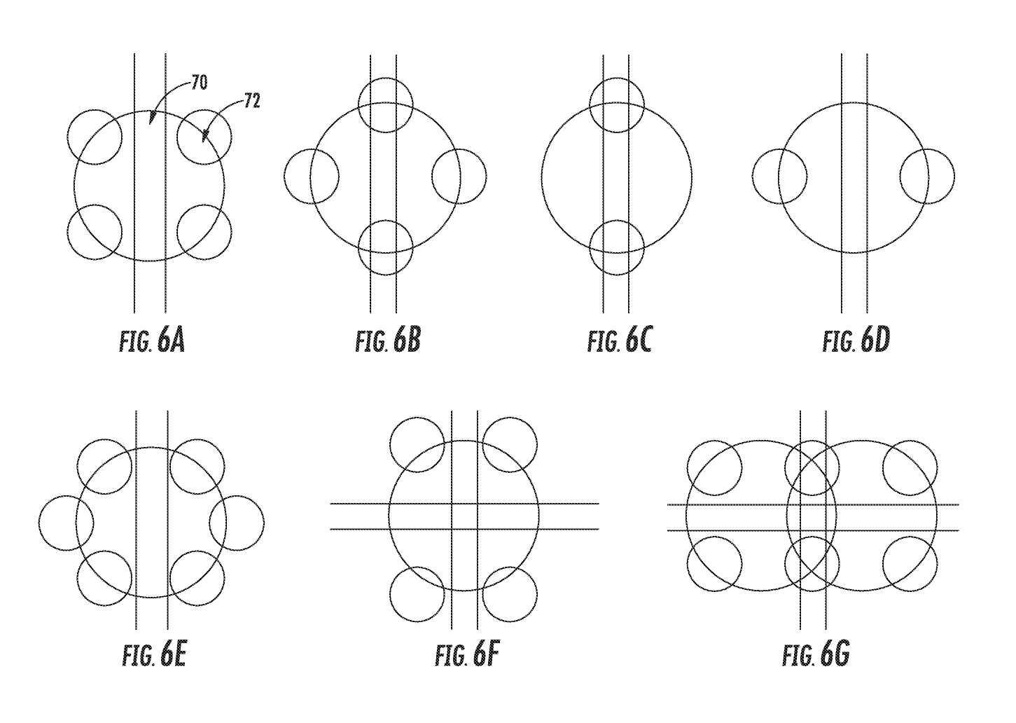

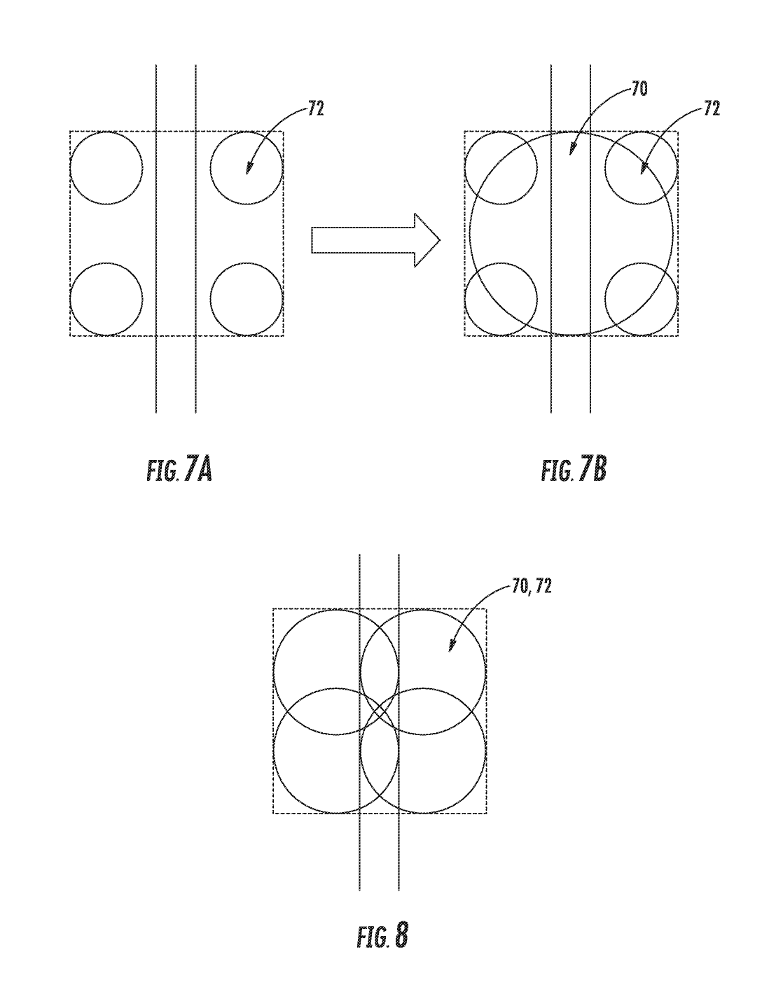

FIGS. 6A-6G illustrates exemplary drilling patterns that can be utilized in conjunction with/provided by the present invention. The commonality among these drilling patterns is that they each include a major bore 70 that is drilled into/across the joint and one or more minor bores 72 that are drilled into/across the joint, overlapping the major bore 70. In practice, the minor bores 72 can be drilled first with the drill guide 60 (FIGS. 4A-4C and 5A-5B) inserted into the drill guide tube 40 (FIGS. 2A-2C, 3, and 5A-5B), which is inserted into the portal tube 10 (FIGS. 1A-1C, 3, and 5A), with the major bore 70 drilled second having the drill guide 60 removed from the drill guide tube. Alternatively, the major bore 70 can be drilled first without the drill guide 60 inserted into the drill guide tube 40, with the minor bores 72 being drilled second with the drill guide 60 inserted into the drill guide tube 40 inserted into the portal tube 10. Multiple major bores 70 can also be utilized, with the appropriate drill guide tube 40 and/or adjustment of the position of the portal tube 10 and/or drill guide tube 40 between the drilling of each major bore 70 and/or the minor bores 72. The resulting drilling pattern can thus form a rough square recess (illustrated), a rough diamond recess (illustrated), a rough H recess (illustrated), a rough rectangle recess (illustrated), etc. into which a corresponding implant can subsequently be impacted and press fit, as described below. It will be readily apparent to those of ordinary skill in the art that, using a variety of sizes and locations of apertures, a wide array of implant receiving bores can be created in terms of size and shape.

FIGS. 7A-7B is a series of schematic diagrams of another exemplary drilling pattern that can be utilized in conjunction with/provided by the present invention, highlighting that the major/minor bores 70 and 72 can be drilled in any order to form the predetermined void shape that is suitable for receiving the implant (not illustrated). Some of the schematic diagrams show the minor bores 72 positioned in rectangular arrays about the major bore 70.

FIG. 8 is a schematic diagram of a further exemplary drilling pattern that can be utilized in conjunction with/provided by the present invention, highlighting that the major/minor bores 70 and 72 may all be similarly sized to form the predetermined void shape that is suitable for receiving the implant (not illustrated).

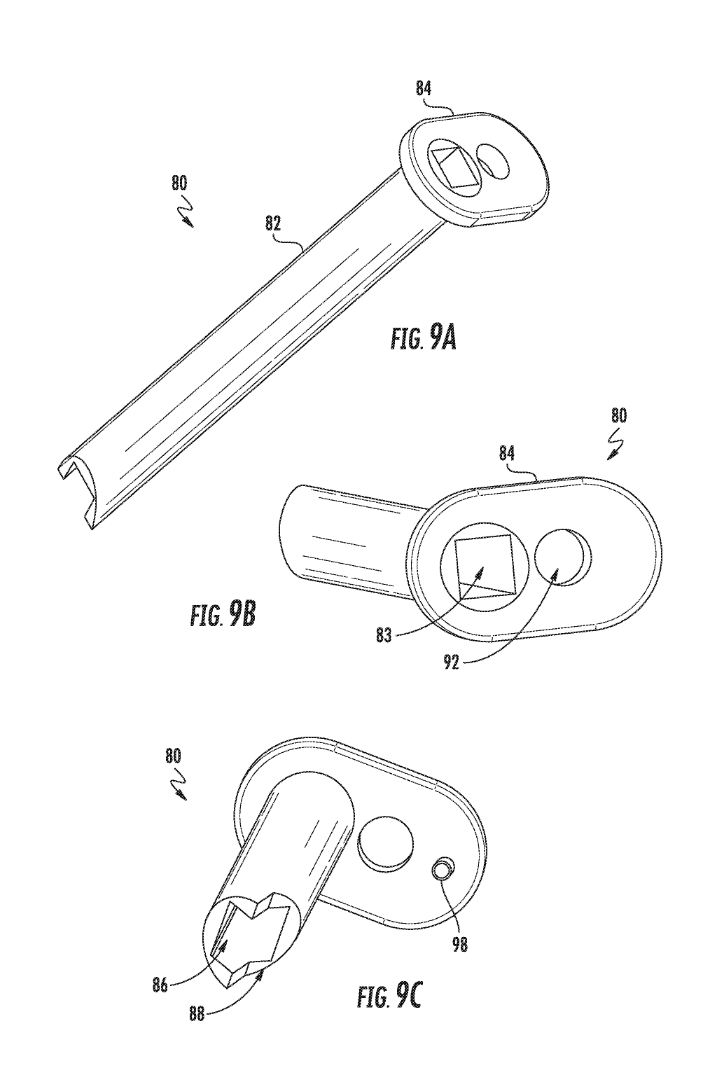

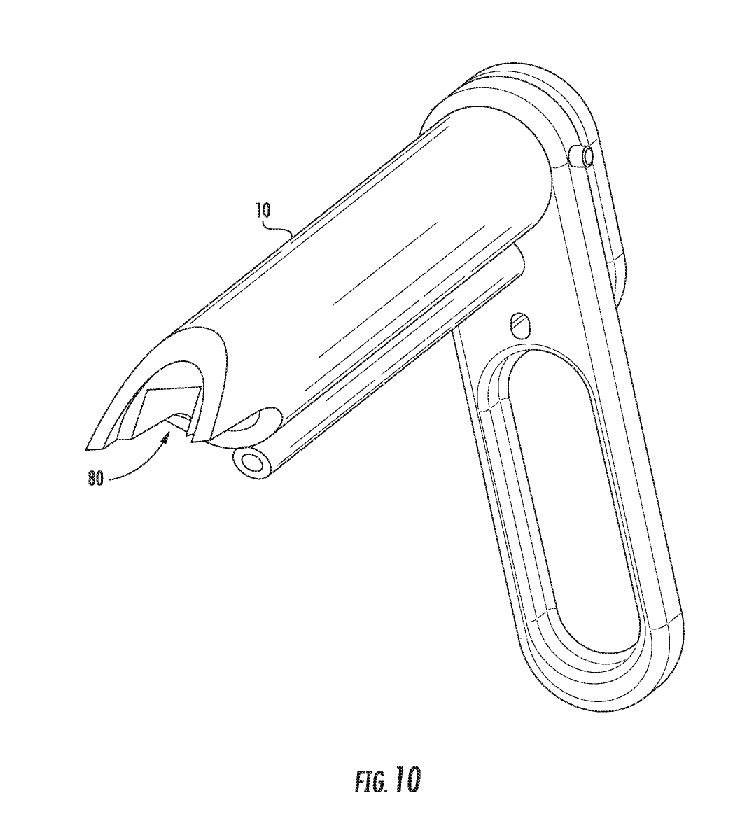

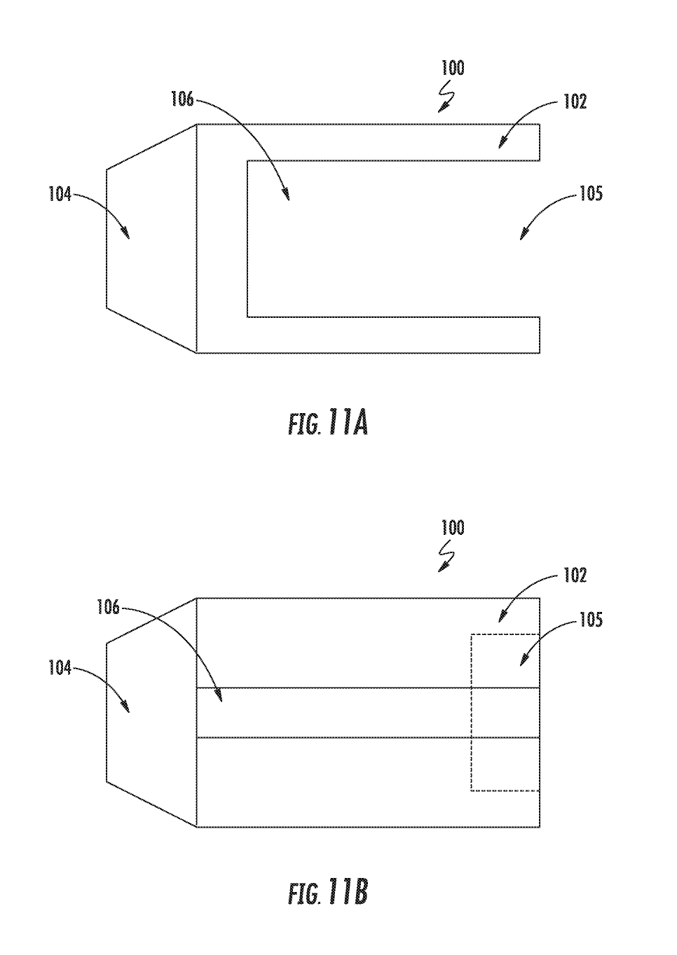

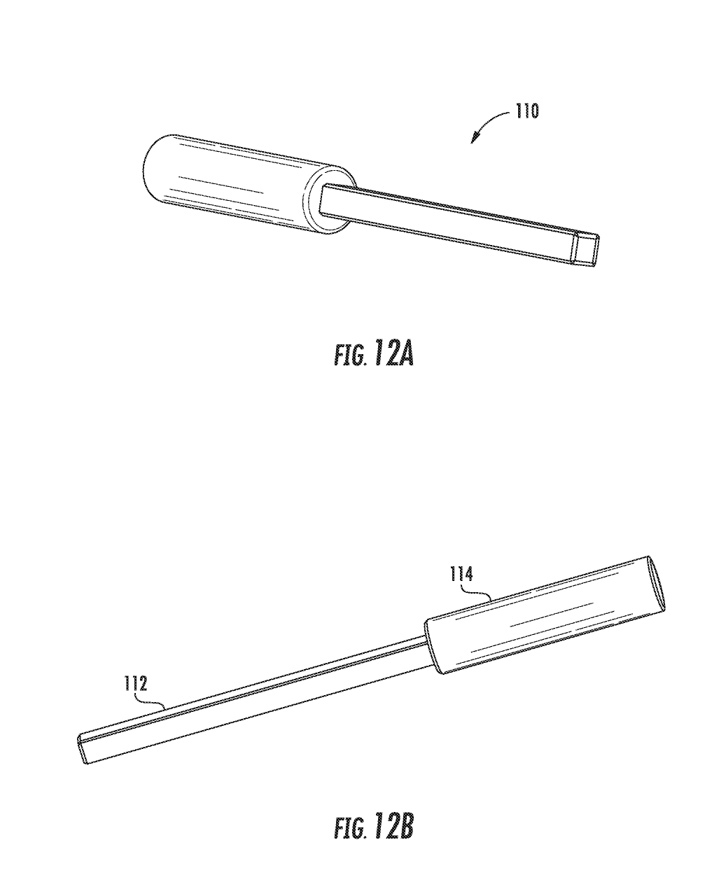

Referring now specifically to FIGS. 9A-9C, in one exemplary embodiment, the implant guide tube 80 includes a cannulated implant tube 82 and a handle 84 coupled to the proximal end of the implant tube 82. In this exemplary embodiment, the implant tube 82 defines an implant aperture 83 having a generally square shape through which a corresponding generally square implant is disposed in a generally square implant receiving bore formed as described above. It will be readily apparent to those of ordinary skill in the art that other shapes could be used equally. The handle 84 is used to manipulate the implant tube 82, as well as to secure the implant tube 82 inside the access tube 12 (FIGS. 1A-1C) after the drill tube 42 (FIGS. 2A-2C) is removed subsequent to drilling, for example. The distal end of the implant tube 82 includes an angled end 86, optionally including a cut away 88, that is configured and shaped to engage the sacroiliac joint, or another joint, such that the implant tube 82 is held in proper alignment, without penetrating too deeply. The angled end 86/cut away 88 of the implant tube 82 is preferably conformal with the angled end 16/cut away 18 (FIGS. 1A and 3) of the access tube 12 when the implant tube 82 is inserted into the access tube 12. Preferably, the implant tube 82 is made of a surgically compatible metal or plastic, and has a length of between about 120 and about 150 mm, an external diameter of between about 15 and about 20 mm, and an internal width of between about 7 and about 12 mm. The handle 84 is a generally paddle shaped component and may include a recess, finger contours, etc. The handle 84 includes a first port 92 that is configured to receive the shoulder stop of the guide pin 24 (FIG. 1A). The handle 84 also includes a pin 98 that is configured to engage the corresponding second port 28 (FIG. 1B) of the handle 14 (FIGS. 1A-1C and 3) of the access tube 12 when the implant tube 82 is inserted into the access tube 12, thereby preventing the relative rotation of the implant tube 82 with respect to the access tube 12. In general, the implant tube 82 acts as an implant alignment and insertion guide relative to the joint in an open surgical procedure or a minimally-invasive surgical procedure.

FIG. 10 illustrates the portal tube 10 and the implant guide tube 80 in an assembled configuration.

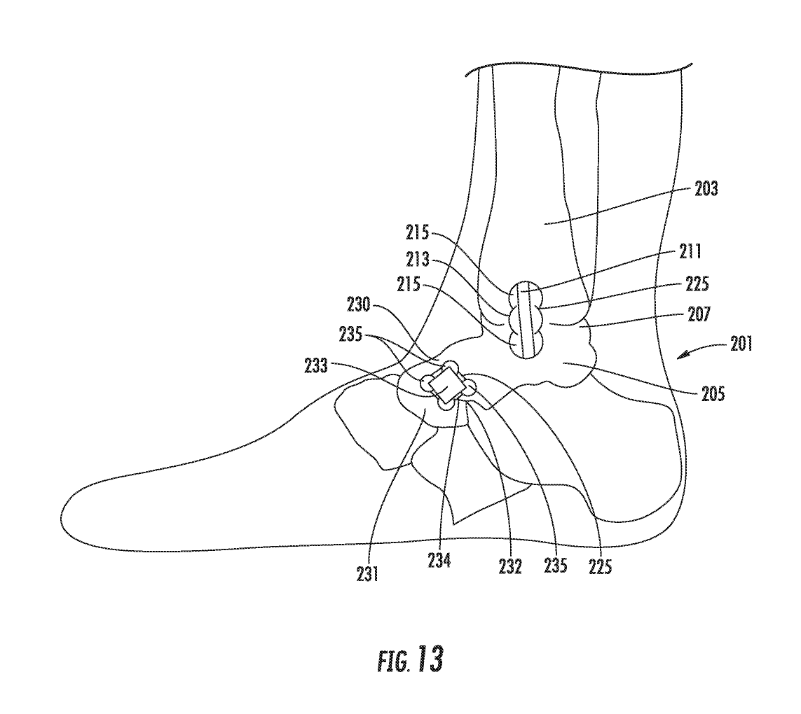

Referring now specifically to FIGS. 11A-11B, in one exemplary embodiment, the implant 100 of the present invention includes a body portion 102 and, optionally, a tapered tip portion 104. The tapered tip portion 104 aides in translating the implant into and through the implant guide tube 80 and press fitting the implant 100 into the bore prepared by the drilling techniques described above. Once placed, the body portion 102 promotes stabilization/fusion of the sacroiliac or other joint, and may also optionally provide or secure a desired degree of translation and/or distraction of the joint. Accordingly, it is desirable that the implant 100 have roughly the same shape perpendicular to its primary axis as the bore prepared by the drilling techniques described above. The implant may be made of a surgically compatible metal or plastic, bone allograft material, or the like. Typically, the implant 100 has a length of between about 17 and about 25 mm and a width or thickness of between about 7 and about 15 mm. Optionally, the body portion 102 defines one or more internal or external voids or channels 106 that is/are configured to receive bone graft material, thereby promoting bony fusion of the joint. Optionally, the body portion 102 also defines one or more internal or external voids or channels 105 that is/are configured to receive a placement/impaction tool, such as that described below. Finally, the body portion 102 may include one or more external friction structures, or the like (not shown) for preventing the implant 100 from backing out of the bore prepared by the drilling techniques described above. Those friction structures may include, but should not be limited to, knurling, directional notches or teeth, rough or porous surface finishes and the like. In practice, the implant 100 may be “over impacted” into the bore prepared by the drilling techniques described above, such that a bone “cap” of bone fusion promoting material or the like can be disposed on top of the implant 100. It should be noted that, in this regard, bone fusion promoting material can be placed in the bore before and/or after the implant 100 is impacted into the bore.

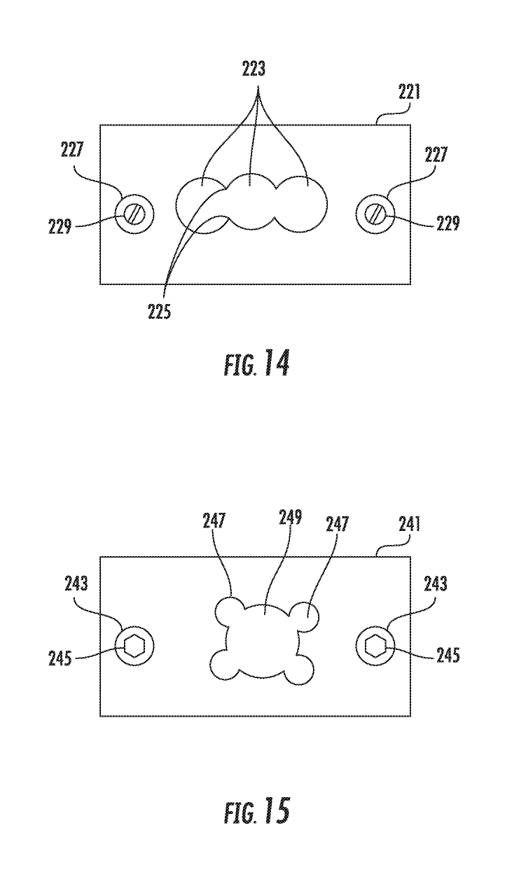

Referring now specifically to FIGS. 12A-12B, in one exemplary embodiment, the implant impaction tool 110 of the present invention includes an elongate body portion 112 that substantially conforms to the internal shape of the implant guide tube 80 (FIGS. 9A-9C and 10) and a handle portion 114. The implant impaction tool 110 is used to translate the implant 100 (FIGS. 11A-11B) through the implant guide tube 80 and press fit the implant 100 into the bore prepared by the drilling techniques described above. Accordingly, the elongate body portion 112 has a length of between about 135 and about 150 mm, and the handle portion or a separate shoulder stop may act as a penetration depth limiter.

FIGS. 13-19 illustrate a second embodiment of the present invention that is particularly directed for use in fusing a highly articulated joint such as an ankle, wrist, or the like. A highly articulated joint is one that allows one bone to move relative to another bone in a pivoting manner of at least 20°. This procedure is often referred to as arthrodesis. It involves artificially causing joint ossification between two bones, one each on opposite sides of the joint, by surgery. Such a procedure tends to be one of last resort when other treatments are not effective. Such a procedure can be done by using bone from elsewhere in the patient's body (autograft) or using donor bone (allograft) from a bone bank. The graft itself forms new bone (Ostia was inducted), as well as acting as a scaffold or matrix to new bone growing from the bones being bridged (ostia conductive). A synthetic bone substitute can also be used. A metal implant can be attached to bridge the two bones to hold them together in a position which favors bone growth to effect fusion of the joint over time and its immobilization. Healing can take months to over a year. It is typical to remove material at the joint, such as cartilage and even some bone material, prior to fixing the position of one bone relative to the other bone on the opposite side of the joint.

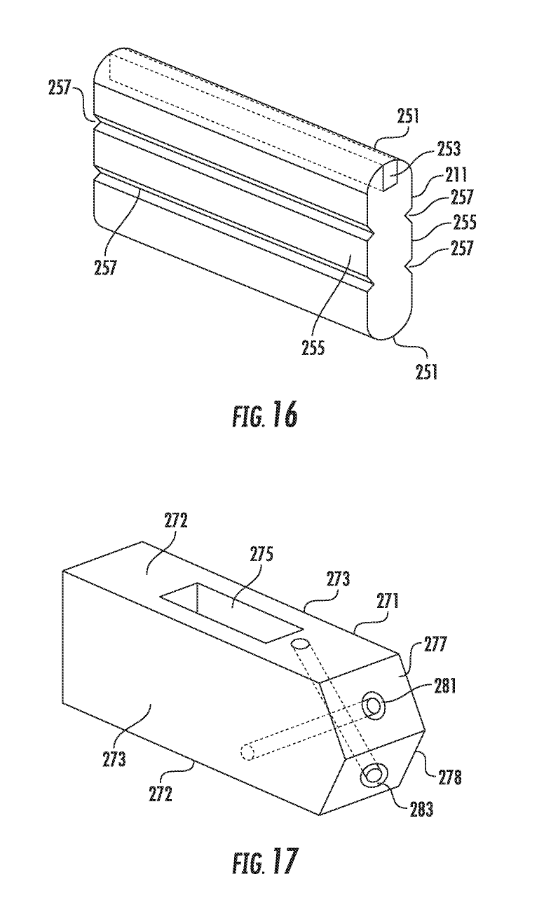

FIG. 13 illustrates a second embodiment of the present invention. It shows, in side view, a highly articulated (or mobile) joint such as an ankle joint 201 including a tibia 203 and a talus 205 (also called astragalus). It is to be understood that while an ankle joint 201 is described, any other articulated joint, including semi mobile joints (those that articulate less than) 20° can be treated according to the present invention; such articulated joints including feet, knees, wrists, cervical spine and the like without departing from the scope of the art. The ankle joint 201 pivots at 207, at a space between the adjacent bones which contains cartilage. As shown, an implant 211 is secured in a machined major bore 213, with the bore 213 bridging the pivot area 207 and having a portion thereof in each of the bones, the tibia and the talus 203, 205 respectively. The bore 213 as shown is formed by a plurality of overlapping round bores 215. Preferably, there are at least three overlapping bores 215. There are spandrels 225 between adjacent overlapping bores 215 forming the bore 213, as seen in FIG. 14. As seen in FIG. 13, the bores 215 are in line; however, it is to be noted that any suitable bore configuration can be provided, as for example, those seen in FIGS. 6A-6G, 7A-7B and 8, as described above. For in line bores of approximately the same diameter, the overlap between adjacent bores is preferably at least about 90° of arc and less than about 140° of arc.

FIG. 13 also illustrates a second implant positioned at a semi mobile joint 230 (a semi mobile foot joint) between the talus 205 and the navicular 231. The joint 230 pivots at 232. As shown, an implant 233 is secured in a machined major bore 234, with the bore 234 bridging the pivot area 232 and having a portion thereof in each of the bones, the navicular 231 and the talus 205. The major bore 234, as shown, is formed by a plurality of overlapping round bores 235. Preferably, there are at least three overlapping bores 235. There are spandrels 225 between adjacent overlapping bores 235 forming the major bore 234, as seen in FIG. 14. The bores 235 are in line; however, it is to be noted that any suitable bore configuration can be provided, as for example, those seen in FIGS. 6A-6G, 7A-7B and 8 as described above. The implant 233 can be similar in construction to the implant 211 described herein.

FIG. 14 illustrates a first drilling guide 221 that can be made out of a surgically compatible material such as a polymer and/or metal such as stainless steel. The thickness of the guide 221 is sufficient to ensure that the machining tool, such as a drill bit, will not drift excessively during the machining of the bore 213. The drilling guide 221 has a plurality of overlapping round apertures 223 having spandrels 225 therebetween. The guide 221 is also provided with through apertures 227 which are sized and shaped to receive therethrough mechanical fasteners, such as screws 229 used to removably attach the guide 221 to the tibia 203 and the talus 205, holding the guide 221 in a secure position, bridging the joint 201 between the tibia 203 and talus 205.

FIG. 15 illustrates a second form of drilling guide 241. It has through apertures 243 positioned for securement of the guide 241 to bones on opposite sides of the joint 201. The apertures 243 are sized and shaped to receive therethrough mechanical fasteners 245 that are used to removably attach the guide 241 to the bones on opposite sides of the joint 201, as described above for the guide 221. The guide 241 has a plurality of overlapping round apertures 247 and 249. As seen in this configuration of guide 241, the aperture 249 is a large central aperture, while the apertures 247 overlap the periphery of the aperture 249. In use, it is preferred to first machine four periphery bores 235, followed by machining of a central bore 234. Machining of the bores can be done with a suitable drill bit inserted through apertures 247, 249 and 223. The guide 241 can be made of a suitable surgically compatible material, such as a polymer and/or metal such as stainless steel, and the thickness of the guide 241 is sufficient to ensure that the machining tool, such as a drill bit, will not drift excessively during the machining of the bores.

In a preferred embodiment, when the bore 213, such as in FIG. 13, is machined, it should be generally centrally located over the abutting end surfaces of the bones 203, 205 forming the pivot 207. Thus, the bore 213 bridges the pivot 207.

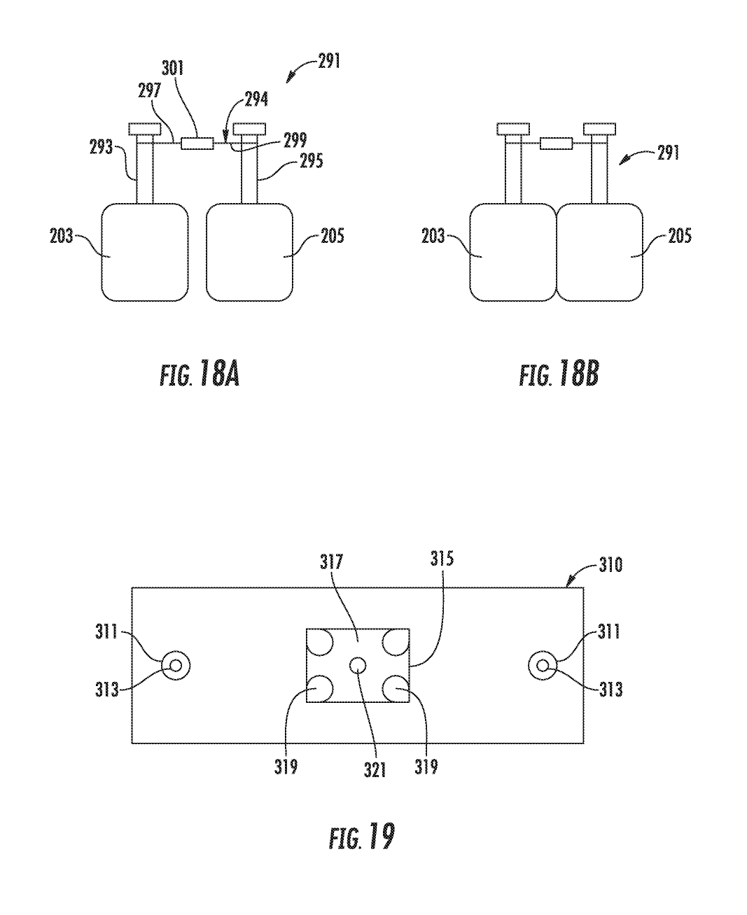

FIG. 16 shows a first form of implant 211. As shown, the implant 211 is generally rectangular in transverse cross-section. As further illustrated, the implant 211 has rounded minor side edges 251 that can correspond in size and shape to the top and bottom edges of the bore 213. It is to be noted that the side edges 251 can also be generally planar to provide a gap between the side edges 251 and the top and bottom edges of the bore 213. It is also to be understood that the implant 211 can be provided with recesses and/or pockets like a recess 253 along one side edge 251, which can be used to contain graft growth inducing material for insertion into the bore 213. The recess 253 is shown in dashed lines since it is optional. The implant 211 has two main planar surfaces 255 on opposite sides of the implant 211 that will engage spandrels 225 to provide a friction fit of the implant 211 within the bore 213. As shown, the implant 211 in FIG. 16 can be provided with the grooves 257 that can each receive a respective spandrel 225 therein to help hold the ends of the bones 203, 205 in a desired relative location, for example, in contact or slightly spaced from one another as desired. The grooves 257 extend between the opposite ends of the implant 211. The implant 211, when installed, helps prevent relative motion between the bones 203, 205 to assist in the bone graft growth. The bore patterns described for the first embodiment can also be machined providing a major bore 70 and a plurality of minor bores 72.

FIG. 17 illustrates a second form of implant and is designated 271. It can have any suitable exterior shape and size, and can be provided with grooves 257 and can have planar side edges 272 or rounded side edges, like the edges 251, and a pair of main planar surfaces 273. The implant 271 can also be provided with a pocket 275 that can be provided with bone graft material for insertion into the machined bore 213. As shown, the implant 271 has on one end, and optionally on both ends, a pair of beveled surfaces 277, 278. A through aperture 281 starts on the surface 277, while a through aperture 283 starts on the surface 278, each extending through to a respective side surface 272. A mechanical fastener, such as a screw (not shown), can be inserted through the respective apertures 281, 283 and used to permanently attach the implant 271 to one or both of the bones 203, 205 after installation of the implant.

The implants 211 and 271 can be made of an implant compatible material such as bone, a polymer and/or metal (including metal alloy) or the like.

FIGS. 18A, 18B illustrate a device (temporary fixator) 291 adapted for positioning and retaining adjacent bones, such as the bones 203 and 205, in positions relative to one another. Both FIGS. 18A, 18B schematically show the device 291, with FIG. 18A showing the bones 203, 205 in a spaced apart relationship, while FIG. 18B shows the bones in contact with one another. The device 291 includes a pair of posts 293 and 295, each secured to a respective bone 203, 205. The attachment can be by each of the posts 293, 295 having a threaded end threadably secured to the respective bone. A winch assembly 294 is operably coupled to the posts 293, 295, and is operable for pulling the posts toward one another, and hence, also moving the respective bones 203, 205 toward one another. The winch assembly 294 can be any suitable device, such as a pair of cables 297, 299 coupled with a tensioning device 301, such as a ratchet or a turnbuckle, that upon activation will shorten the cables 297, 299 and move the posts 293, 295 closer to one another, and hence, the bones as seen in FIG. 18B. The winch assembly 294 could also include a screw rotatably mounted in one post 293 and threadably engaged with the other post 295. Once the bones 203, 205 are in the proper position, then the drilling guide 221 or 241 can be attached in position, bridging the joint 201/pivot 207 and the bore 213 machined as by drilling into the bones 203, 205. After the bore 213 is formed, the guide 221 or 241 can be removed and the implant 211 or 271 can be installed, such as by press fitting, hammering, screwing or the like.

FIG. 19 illustrates a third embodiment of drilling guide 310 in top plan view. It has through apertures 311 positioned for securement of the guide 310 to bones on opposite sides of the joint 201. The apertures 311 are sized and shaped to receive therethrough mechanical fasteners 313 that are used to removably attach the guide 310 to the bones on opposite sides of the joint 201, as described above for the guide 221. The guide 310 has a through aperture 315 adapted to receive a removable insert 317 therein. The insert 317 and aperture 315 are shown as being generally rectangular in transverse cross-sectional shape. A partially round through aperture segment 319 is positioned at each corner of the insert 317. A pilot through aperture 321 is located in the insert 317 and is preferably centrally located. The pilot aperture is machined in the joint 201, preferably where the bones 203, 205 meet. A guide pin (not shown) can then be inserted into the machined bore to help locate or position a new insert 317 or the guide 310 for its location before machining the plurality of minor bores in the bones. Minor bores can then be machined into the bones 203, 205 using the aperture segments 319 as guides. The insert 317 can then be removed and a new insert installed, allowing guided drilling of a major bore in one or both of the bones 203, 205 overlapping the minor bores.

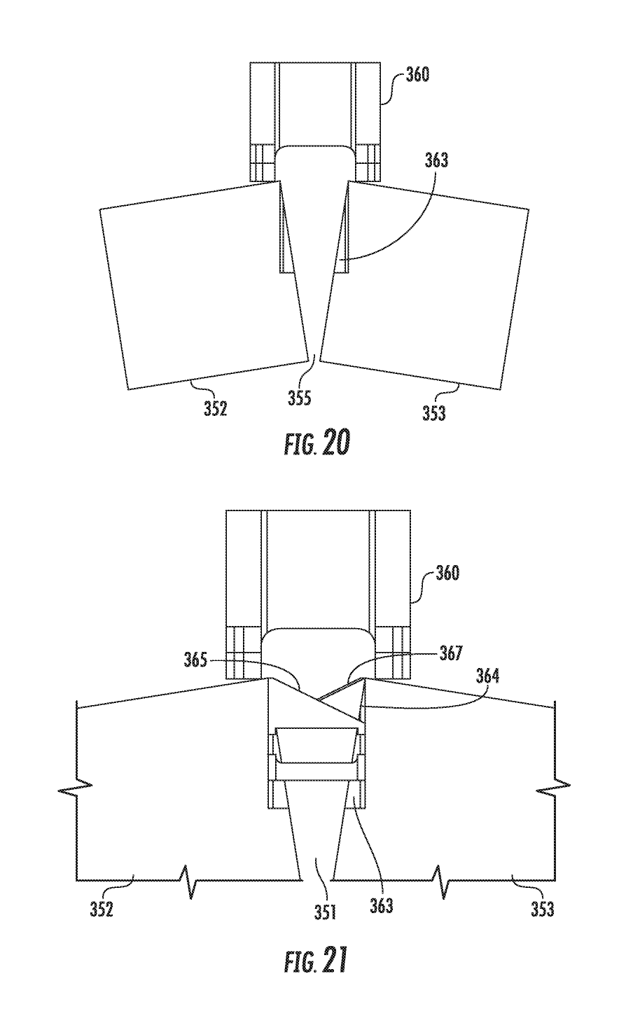

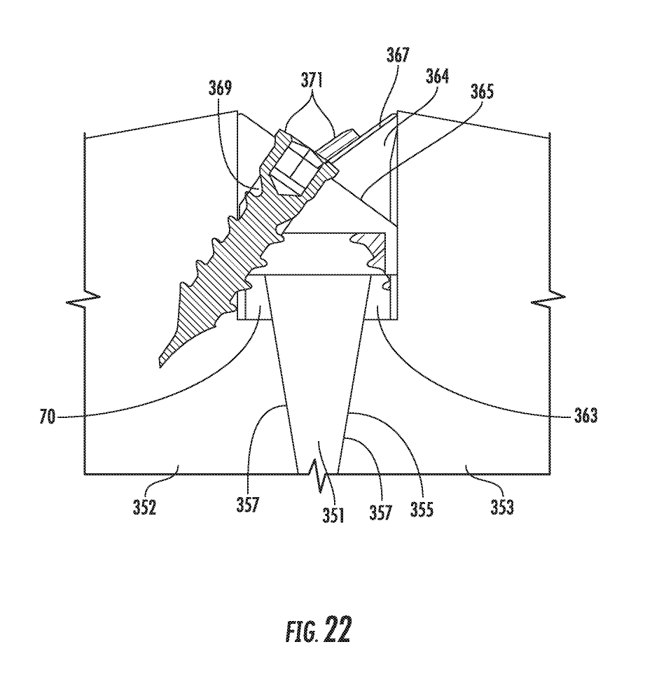

FIGS. 20-22 illustrate another embodiment of the present invention that will use a wedge shaped implant component 351 between adjacent bones 352, 353 at a joint 355 therebetween. In one example, the bones 352, 353 can be between adjacent vertebrae at a cervical spine joint or at other joints in the spine. The implant component 351 includes a pair of somewhat opposed main generally planar surfaces 357. A drilling guide 360 is positioned to bridge the joint 355 and can be any suitable drilling guide, such as the drilling guides 60, 221, 241 and 310 as described above. Also as described above, a void 363 is machined in the adjacent bones 352, 353, also as described above, and can utilize the above described major bore 70 and minor bores 72. As illustrated, the joint 355 has a wedge shape that is to be maintained after the fusion process is complete. As best seen in FIGS. 21, 22, the implant has two components: the wedge shaped component 351 and a retainer component 364. The retainer component 364 is similar in some regards to the implant 271 described above. It has a pair of beveled surfaces 365, 367, each with a through aperture 369 adapted to receive therethrough a respective mechanical fastener 371, such as a screw, operable to mechanically fasten the retainer component 364 to both of the adjacent bones 352, 353 and thereby retain the implant 351 in place. The implant 351 can function not only as a bone position retainer, but also as a scaffold to help induce bone fusion. Similar to the above described implants, the implant component 351 can be made of an implant compatible material such as bone, a polymer, and/or metal as known in the art.

In the method of the present invention, a patient is anesthetized. Thereafter, the surgeon will make the appropriate opening to the joint as determined by the desired minimally invasive procedure or the open surgery procedure. The surgeon can then remove undesirable materials removed therefrom, as is known in the art. If needed, the bones 203, 205 can then be moved to the appropriate position relative to one another as described above regarding the device shown in FIGS. 18A-18B. The appropriate drilling guide, such as guide 211, is then installed in the bore 213, or another appropriate bore is machined as described above. The desired implant, such as implant 211, is then installed. If desired, material to assist in forming the bone graft can be added also as described above. The surgical site is then closed, and other devices, such as a cast or the like, can be used to provide additional rigidity for the joint during healing.

All patents and publications mentioned in this specification are indicative of the levels of those skilled in the art to which the invention pertains.

It is to be understood that while a certain form of the invention is illustrated, it is not to be limited to the specific form or arrangement herein described and shown. It will be apparent to those skilled in the art that various changes may be made without departing from the scope of the invention, and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A joint fixation method, comprising:
machining a plurality of overlapping first bores in a pair of bones, each being on opposite sides of an articulated joint, said overlapping first bores forming a major bore bridging a pivot area of the joint, said overlapping bores having spandrels therebetween; and
securing an implant in the major bore and bridging said joint.

2. The method of claim 1, wherein said overlapping first bores are in line.

3. The method of claim 2, there being at least one said first bore in each of said bones.

4. The method of claim 3, wherein said implant engaging said spandrels.

5. The method of claim 4, wherein said spandrels being received in grooves in said implant.

6. The method of claim 1, wherein said implant being at least partially secured by friction.

7. The method of claim 1, wherein said implant being at least partially secured by a mechanical fastener.

8. The method of claim 1, wherein said first bores including at least one major bore and a plurality of overlapping minor bores associated therewith.

9. The method of claim 8, wherein said minor bores being positioned in a rectangular array.

10. The method of claim 1, including temporarily securing a winch assembly to said pair of bones and moving said bones to a desired relative location prior to machining said first bores.

11. The method of claim 1, including inserting material at said joint to assist in forming a bone graft.

12. The method of claim 11, wherein said inserted material is carried by said implant.

13. The method of Claim 1, wherein the joint being a highly articulated joint.

14. The method of claim 13, wherein the joint including one of an ankle joint, a knee joint and a wrist joint.

15. The method of claim 1, wherein the joint being a semi mobile joint.

16. The method of claim 15, wherein the joint being a cervical spine joint.

17. The method of claim 16, wherein the joint being foot joint.
